(19) United States
(12) Patent Application Publication
(10) Pub. No.: US 2021/0177526 A1
(43) Pub. Date: Jun. 17, 2021

(54) METHOD AND SYSTEM FOR SPINE TRACKING IN COMPUTER-ASSISTED SURGERY

(71) Applicant: ORTHOSOFT ULC, Montreal (CA)

(72) Inventors: Andreanne GOYETTE, Montreal (CA); Ramnada CHAV, Laval (CA); Karine DUVAL, Montreal (CA)

(21) Appl. No.: 17/123,260

(22) Filed: Dec. 16, 2020

Related U.S. Application Data
(60) Provisional application No. 62/948,494, filed on Dec. 16, 2019.

(51) Int. Cl. A61B 34/20; A61B 34/30; A61B 34/00; A61B 17/16

(52) U.S. Cl. CPC A61B 34/20; A61B 34/30; A61B 2090/363; A61B 17/1671; A61B 34/25

(57) ABSTRACT

A method for spine tracking in computer-assisted surgery, the method includes: obtaining, at a computer-assisted surgical system, at least one image of at least part of the spine and at least one surgical device; determining, at the computer-assisted surgical system, a three-dimensional position and orientation of the at least one surgical device relative to the spine from the at least one image to create a referential system; tracking, at the computer-assisted surgical system, the at least one surgical device altering a first vertebra of the spine for attachment of a spinal screw to the first vertebra, in the referential system; and tracking, at the computer-assisted surgical system, the spine in the referential system with a trackable reference attached to the spinal screw of the first vertebra.

### CROSS-REFERENCE TO RELATED APPLICATION

The present application claims the priority of U.S. Patent Application No. 62/948,494, filed on Dec. 16, 2019 and incorporated herein by reference.

### TECHNICAL FIELD

The present disclosure relates generally to computer-assisted surgery, and, more particularly, to methods, systems, and devices for spine tracking in computer assisted surgery.

### BACKGROUND OF THE ART

Traditional spinal surgical operations are invasive, often requiring large incisions which, while necessary to achieve sufficient spinal exposure, result in extended patient trauma and post-operative pain. Computer-assisted image guided surgical instrument navigation is typically used wherever possible in an effort to reduce the invasiveness of spinal surgery. Nevertheless, it is still desirable to reduce the invasiveness of spinal surgery.

As such, there is a need for improved methods, systems and devices for spine tracking in computer-assisted surgery.

### SUMMARY

The present disclosure is generally drawn to methods, systems, and devices for spine tracking in computer-assisted surgery.

In one aspect, there is provided a method for spine tracking in computer-assisted surgery, the method comprising: obtaining, at a computer-assisted surgical system, at least one image of at least part of the spine and at least one surgical device; determining, at the computer-assisted surgical system, a three-dimensional position and orientation of the at least one surgical device relative to the spine from the at least one image to create a referential system; tracking, at the computer-assisted surgical system, the at least one surgical device altering a first vertebra of the spine for attachment of a spinal screw to the first vertebra, in the referential system; and tracking, at the computer-assisted surgical system, the spine in the referential system with a trackable reference attached to the spinal screw of the first vertebra.

In another aspect, there is provided a system for spine tracking in computer-assisted surgery, the system comprising: a processing unit; and a non-transitory computer-readable memory having stored thereon program instructions executable by the processing unit for: obtaining at least one image of at least part of the spine and at least one surgical device; automatically registering a three-dimensional position and orientation of the at least one surgical device relative to the spine from the at least one image to create a referential system; tracking the at least one surgical device altering a first vertebra of the spine for attachment of a spinal screw to the first vertebra, in the referential system; and tracking the spine in the referential system with a trackable reference attached to the spinal screw of the first vertebra.

In another aspect, there is provided an assembly for spine tracking in computer-assisted surgery, the assembly comprising: a spinal screw having a connector; a surgical device including an attachment member for coupling to the spinal screw, and a trackable member coupled to the attachment member, the trackable member including at least one detectable element for being tracked in three-dimensional space by a computer-assisted surgical system, thereby allowing tracking position and orientation of a spine by the computer-assisted surgical system when the attachment member is coupled to the spinal screw implanted in a vertebra of the spine.

### DESCRIPTION OF THE DRAWINGS

Reference is now made to the accompanying figures in which:

FIG. 1A is a perspective view of a surgical device comprising a trackable member, in accordance with an embodiment;

FIG. 1B is a perspective view of the surgical device of FIG. 1A with a variant of the trackable member, in accordance with an embodiment;

FIG. 1C is a cross-sectional view of the surgical device of FIG. 1B from a first perspective, in accordance with an embodiment;

FIG. 1D is a cross-sectional view of the surgical device of FIG. 1B from a second perspective, in accordance with an embodiment;

FIG. 1E is a perspective view of exemplary spinal screws, in accordance with an embodiment;

FIG. 2 is a schematic diagram of a computer-assisted surgical system, in accordance with an embodiment;

FIG. 3 is a flow diagram illustrating an example of a computer-assisted surgical process, in accordance with an embodiment;

FIG. 4 is a flowchart illustrating an example method for spine tracking in computer-assisted surgery, in accordance with an embodiment; and

FIG. 5 is a schematic diagram of an example computing system for implementing at least in part the system of FIG. 2, the process of FIG. 3, and/or the method of FIG. 4, in accordance with an embodiment.

It will be noted that throughout the appended drawings, like features are identified by like reference numerals.

### DETAILED DESCRIPTION

The present disclosure is generally drawn to methods, systems, and devices for spine tracking in computer-assisted surgery (CAS). Imaging of a spine and a reference (e.g., a spinal screw and/or a surgical device having a trackable member) may be obtained and used by a CAS system to determine a three-dimensional (3D) position and orientation of the reference relative to the spine. The reference may be used by the CAS system to determine the position and orientation of the spine and/or to track the position and orientation of the spine during the spinal surgery. The reference may be used by the CAS system to track one or more surgical tools and/or implants relative to the spine during the spinal surgery.

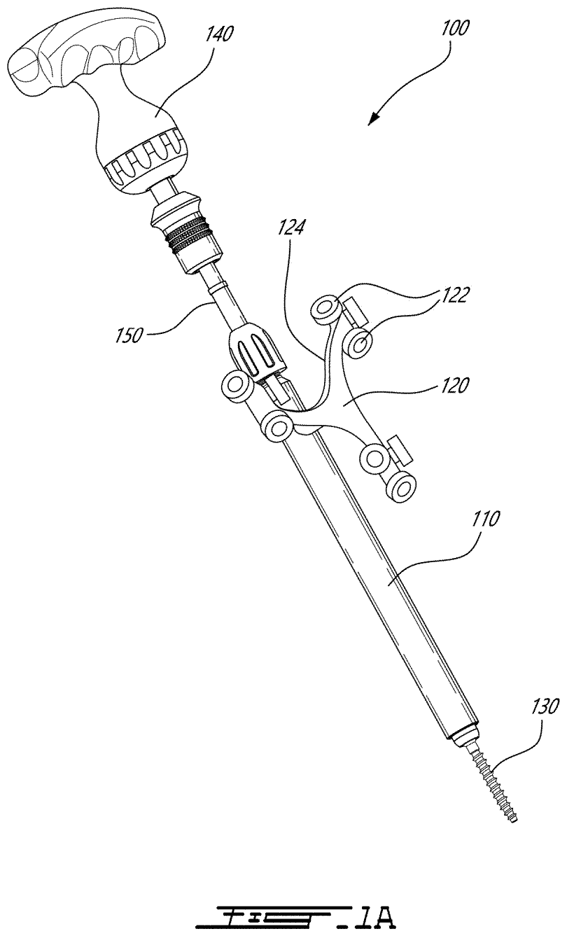

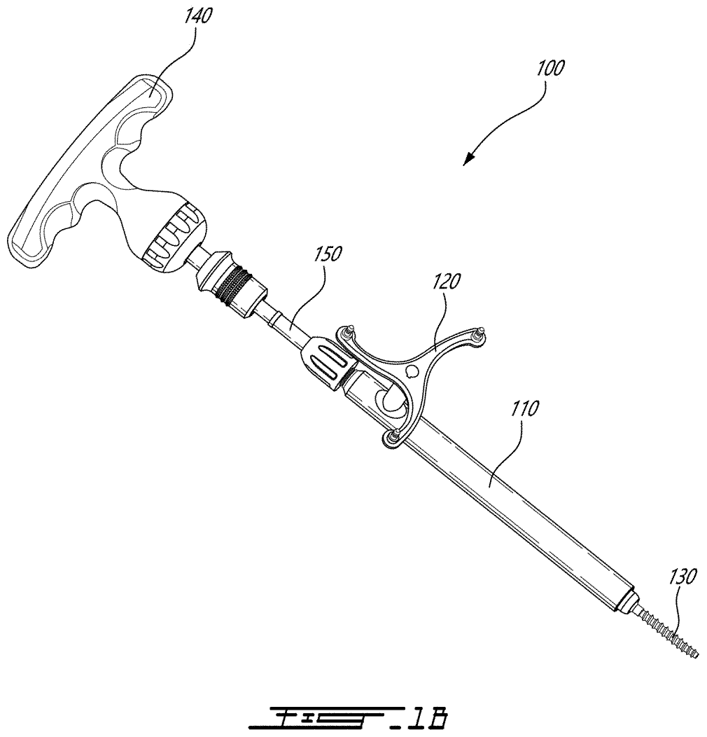

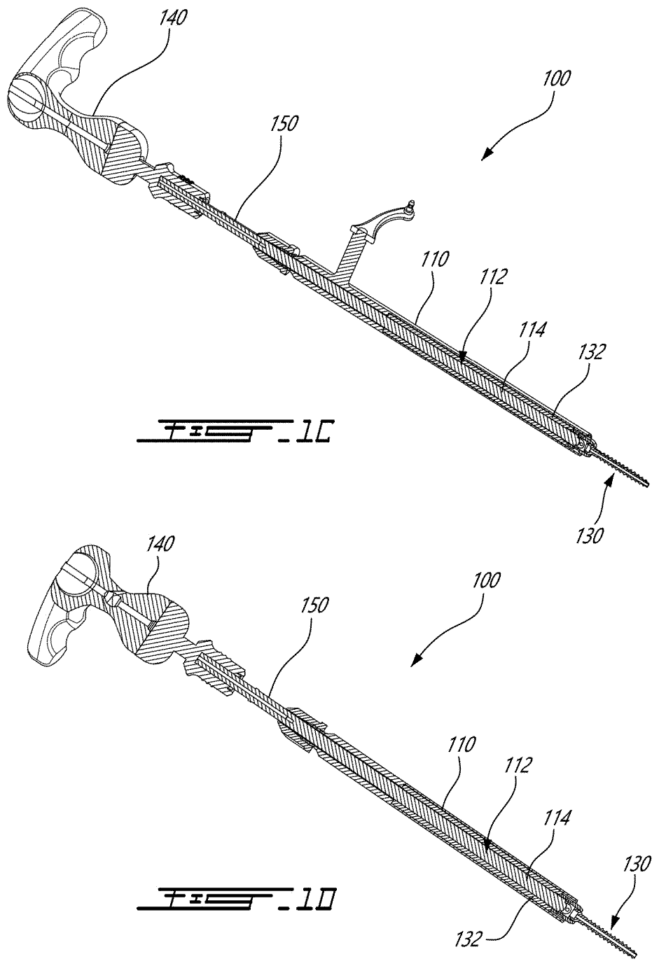

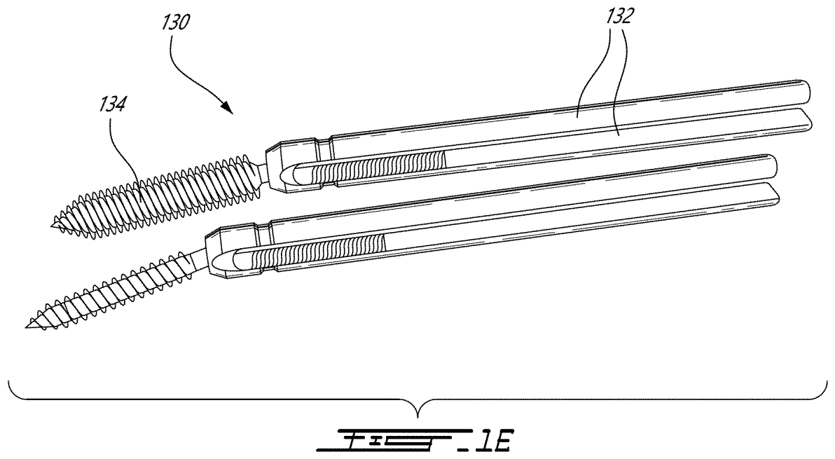

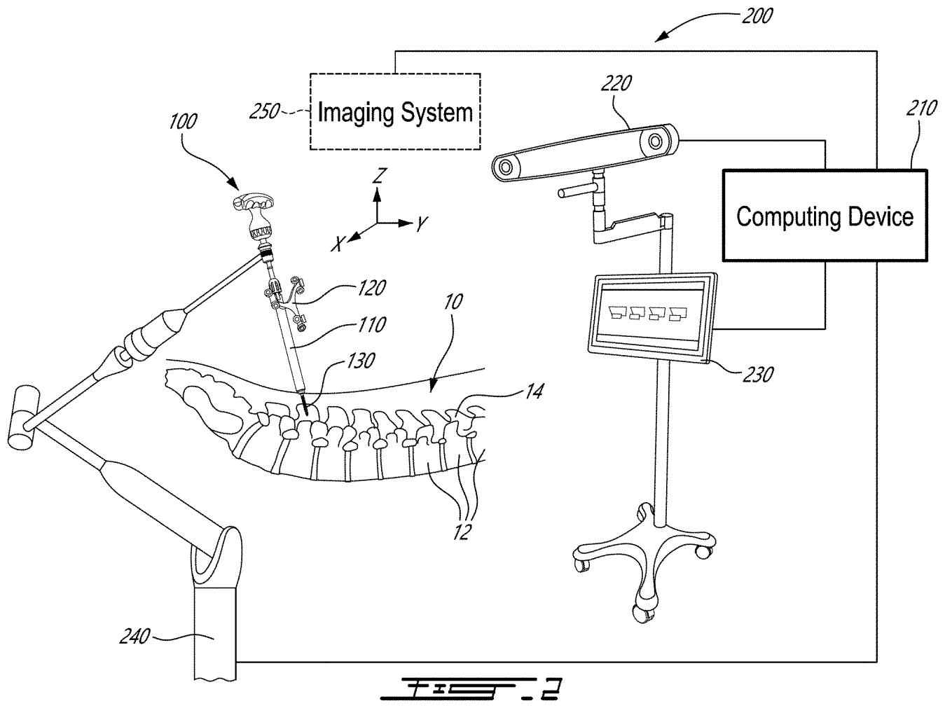

With reference to FIG. 1A, there is illustrated a surgical device 100 for use in a CAS. The surgical device 100 includes an attachment member 110 and may optionally have a trackable member 120. The attachment member 110 is adapted for coupling to a spinal screw 130. More specifically, the attachment member 110 is adapted for being removably attached to a vertebra of a spine via the spinal screw 130 when the spinal screw 130 is implanted in the vertebra. The attachment member 110 may be adapted for removably coupling the screw 130, and preserve its position relative to the screw 130. The attachment member 110 can be decoupled from the screw 130 when not needed. The attachment member 110 may be a cannulated tube, a support rod (e.g., hollow or not) for mounting the trackable member 120 thereon, as shown in FIG. 1. The shape and/or configuration of the attachment member 110 may vary depending on practical implementations.

The trackable member 120 is coupled to the attachment member 110. The trackable member 120 may be removably coupled to the attachment member 110. In other words, the trackable member 120 may be attached to the attachment member 110 when needed during surgery and subsequently removed when not needed. In some embodiments, the trackable member 120 is not removable from the attachment member 110. The trackable member 120 may comprise a plurality of branches 124 each comprising a plurality of detectable elements 122, e.g., circular tokens of retroreflective material. As shown in FIG. 1A, the trackable member 120 may comprise three branches 124, each comprising three detectable elements 122. The number of branches 124 and/or the number of detectable elements 122 of the trackable member 120 may vary depending on practical implementations, and any suitable number of branches and/or detectable elements may be used. In some embodiments, the trackable member 120 is the NavitrackER™ reference marker device provided by Zimmer Biomet. With additional reference to FIG. 1B, the surgical device 100 of FIG. 1A is illustrated with a variant of the trackable member 120 having three detectable elements 122. The shape and/or configuration of the trackable member 120 may vary depending on practical implementations. For instance, instead of the circular tokens shown in FIG. 1A, the detectable elements 122 may be spheres, disks, may have polygonal shapes, etc.

In some embodiments, the surgical device 100 comprises a handle 140. The handle 140 may or may not be removable from the surgical device 100. The handle 140 may be used for turning the surgical device 100 in order to implant the spinal screw 130 into a vertebra. The handle 140 may be connected to a screw driver mechanism 150 adapted for turning (e.g., screwing) the spinal screw 130 coupled to the attachment member 110. With additional reference to FIGS. 10 and 1D, cross-sectional views of the surgical device 100 are illustrated. As shown, the attachment member 110 may be adapted for receiving at least in part the spinal screw 130 therein. More specifically, the attachment member 110 may be hollow so as to have a cavity 112 for receiving tabs of the spinal screw 130 in order to couple the spinal screw 130 to the surgical device 110. Inside the cavity 112, the attachment member 110 may have an elongated rotor component 114. The rotor component 114 is coupled to the screw driver mechanism 150 such that a rotation of the handle 140 causes a rotation of the rotor component 114 relative to the tubular body of the attachment member 110. Therefore, in an embodiment, a user may hold the tubular body of the attachment member 110 or part of the screw driver mechanism 150 while imparting a rotation to the handle 140, such that the rotor component 114 screws the spinal screw 130 into a vertebra, for example.

With additional reference to FIG. 1E, and for being coupled to the rotor component 114, the spinal screw 130 may include a connector such as a bracket that may be defined by two tabs 132 and a screw 134 attached to tabs 132, being elongated in shape. Although the expression tabs is used, other expressions could be used to describe the elongated features that couple to the attachment member 110. The number of tabs 132 may vary depending on practical implementations, and any suitable number of tabs may be used. The spinal screw 130 may vary depending on practical implementations. Some anti-rotation feature may be present between the rotor component 114 and the tabs 132, such as complementary flat surfaces, as one of numerous possibilities. In an embodiment, an inner surface of the attachment member 110 is cylindrical, and the rotor component 114 is a shaft having such complementary flat surfaces. The tabs 132 may be shaped to be snuggly received between the rotor component 114 and space in the inner cavity 112. Therefore, when coupled together as in FIG. 1B, the attachment member 110 and the spinal screw 130 are coaxial. Central axes of the attachment member 110 and the spinal screw 130 have the same orientation, and a trajectory of the spinal screw 130 may be known from a tracking of the longitudinal central axis of the attachment member 110. Other coupling arrangements could be used, for instance with the spinal screw 130 having a socket, and the attachment member 110 having a complementary tool end. Moreover, the attachment member 110 is shown as having an open ended tube housing the rotor component 114. However, the rotor component 114 could be exposed, with the attachment portion of the spinal screw 130, such as the tabs 132, connected to the rotor component 114 for concurrent rotation. A ring could for instance be slid onto the assembly of the rotor component 114 and tabs 132, as a possibility.

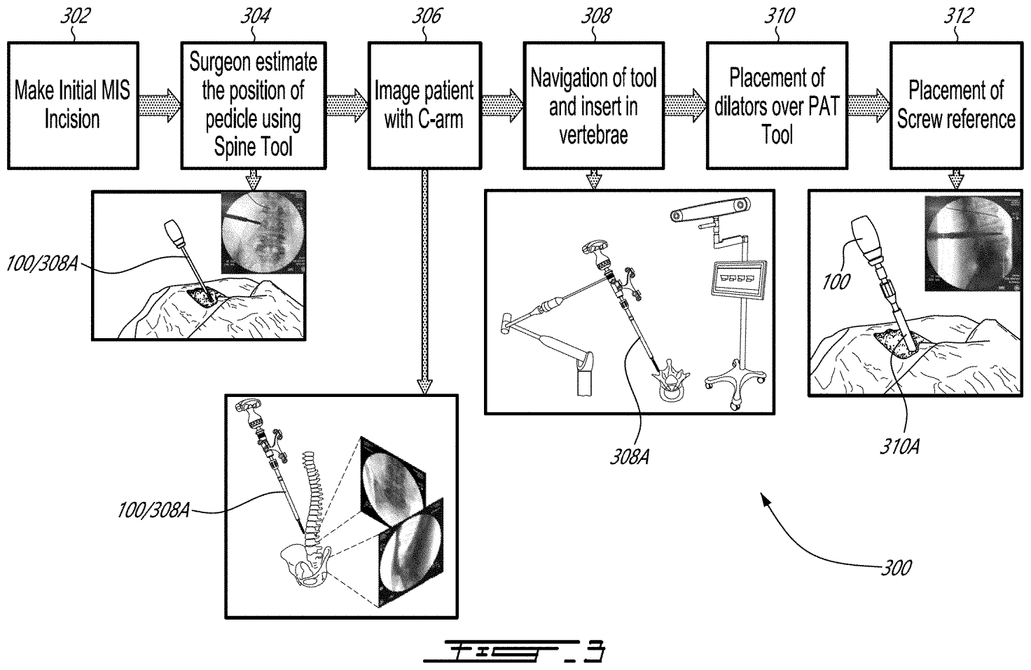

With reference to FIG. 2, there is illustrated a CAS system 200 for use with the surgical device 100. In the illustrated embodiment, the computer-assisted surgical system 200 includes a computing device 210, a tracking camera such as at least one optical sensor 220 for tracking the trackable member 120 and connected to the computing device 210, and a display device 230 connected to the computing device 210. The computing device 210 may be any suitable computing device, such as a desktop computer, a workstation, a laptop computer, a mainframe, a server, a distributed computing system, a cloud computing system, a portable computing device, a mobile phone, a tablet, or the like. The display device 230 may be any suitable display device, for example, such as a cathode ray tube display screen, a light-emitting diode display screen, a liquid crystal display screen, a touch screen, a tablet or any other suitable display device. One or more input device(s) such as a keyboard, a mouse, a touch pad, a joy stick, a light pen, a track ball, a touch screen, and/or any other suitable input device may be connected to the computing device 210 for interacting with a GUI displayed on the display device 230. In embodiments where the display device 230 is a touch screen device, the input device(s) may include the display device 230. In some embodiments, the optical sensor(s) 220 and/or display device 230 may be provided separate from the CAS system 200. The configuration of the CAS system 200 may vary depending on practical implementations.

The optical sensor(s) 220 are for tracking the surgical device 100, and in particular the trackable member 120 if present. The optical sensor(s) 220 may be used to track any other surgical tools and/or implants used during the surgery. Any suitable optical sensor(s) may be used. The optical sensor(s) may be provided as part of an optical system connectable to computing device 210. In some embodiments, the optical sensor(s) 220 are infrared sensors. The sensor(s) 220 may be provided as part of one or more cameras for capturing images of the trackable member 120. In some embodiments, the optical sensor(s) 220 are structured light cameras and/or motion sensing input devices. The optical sensor(s) 220 may be configured to identify and/or track the position and/or orientation of the detectable element(s) 122 of the trackable member 120. With some other tracking modalities, the trackable member 120 may not be required, or may take another form. For example, structured light cameras and/or motion sensing input devices used as the optical sensor(s) 220 may track the surgical device 100 without additional trackable member. The trackable members may be other recognizable features, including patterned labels, etc. Alternatively, the computing device 210 may be able to identify and/or track the detectable element(s) 122 from the data (e.g., images) acquired by the optical sensor(s) 220. Accordingly, the CAS system 200 is able to detect the position and/or orientation of the surgical device 100, such as via the trackable member 120 if present through its movement (e.g., the position of each of the detectable element(s) 122), to then compute a position and/or orientation of the surgical device 100 and/or of the spinal screw 130 using the tracking of the surgical device 100, such as via the trackable member 120, and the geometrical relation between the trackable member 120 (if present), the surgical device 100 and spinal screw 130. Similarly, the CAS system 200 may be able to detect the position and/or orientation any other surgical tools and/or implants used during the surgery. The computing device 210 may obtain the images of the trackable member 120 or any other surgical tools and/or implants from the optical sensor(s) 220 or generate images based on data received from the sensor(s) 220. The images depicting the trackable member 120 or any other surgical tools and/or implants may be displayed on the display device 230 via the GUI.

In some embodiments, the CAS system 200 comprises a robotic arm 240 for controlling the position and orientation of the surgical device 100, though the tracking may also be done in free hand mode as well. Alternatively, the CAS system 200 may be connected to an external robotic arm 240 via the computing device 210. The robotic arm 240 is adapted for holding the surgical device 100. The robotic arm 240 of FIG. 2 is an example of an arm that may be used with the surgical device 100 being connected to an effector end of the robotic arm 240. In an embodiment, the robotic arm 240 may provide 6 DOFs (position and orientation) of movement to the effector end, though fewer or more may be possible. In an embodiment, the robotic arm 240 is used in a collaborative mode, as manipulated by a user, with the possibility to provide some movement constraints, such as blocking the joints of the robotic arm. The robotic arm 240 of FIG. 2 may for example be as described in United States Patent Application Publication No. 2018/0116758, incorporated herein by reference. In such a configuration, the robotic arm 240 may automatically lock in a collaborative mode, once a user is satisfied with the orientation of the surgical device 100.

The position of the robotic arm 240 and the position of the surgical device 100 may also be controlled by interacting with the GUI displayed on the display device 230 via the input device(s). The computing device 210 may accordingly control movements of the robotic arm 240 and the surgical device 100 during the surgery, as requested by the surgeon via the computing device 210 and/or according to an preprogrammed process. In alternative embodiments, the robotic arm 240 may be omitted and the surgeon may manual control the position and orientation of the surgical device 100.

In some embodiments, the CAS system 200 includes an imaging system 250 for obtaining images of anatomy of a patient, for example intra-operatively. Alternatively, the CAS system 200 may be connected to an external imaging system 250 via the computing device 210. As shown in FIG. 2, the anatomy being imaged comprises a spinal column 10, and in particular, a spinal column 10 comprising vertebrae 12, where each vertebra 12 has two pedicles 14. The imaging system 250 may be an X-ray imaging system for providing X-ray images. The X-ray images may be fluoroscope x-ray shots. The imaging system 250 may be a computed tomography (CT) imaging system for providing CT scans. The imaging system 250 may also be an ultrasound imaging system for providing ultrasound images. Any other suitable imaging system may be used. The imaging system 250 may be configured to provide images from different perspectives. For example, the imaging system 250 may provide images from two perspectives, such as a lateral perspective and a posterior perspective. The images may be taken with a C-arm in order to obtain lateral and posterior or anterior images. The images may obtained prior to the spinal surgery and/or intra-operatively during the spinal surgery. For example, images of the spine 10 and of the surgical device 100 may be obtained before alterations to vertebrae. By way of an example, images of the spine 10 and of the surgical device 100 may be obtained intraoperatively with the spinal screw 130 implanted in a vertebra 12. The computing device 210 may obtain the images from the imaging system 250 and the images may be displayed on the display device 230 via the GUI.

The CAS system 200 may be configured to determine the 3D orientation and optionally position of the surgical device 100 relative to the spine 10. Determining the 3D position and/or orientation of the surgical device 100 may include any one or more of the following: determining the position and/or orientation of the attachment member 110, determining the position and orientation of the trackable member 120 and determining the position and orientation of the spinal screw 130, for example relative to a vertebra(e). The images from the imaging system 250 may be processed at the computing device 210 in order to determine the 3D position and/or orientation of the surgical device 100 relative to the spine 10.

The CAS system 200 may determine the position and/or orientation of the surgical device 100 relative to the spine 10 prior to incision of soft tissue, or with a minimally invasive incision that exposes only a part of a vertebra, for example. For example, the robotic arm 240 may be used to hold the surgical device 100 in place for the spinal surgery, at an approximate position and orientation of a desired trajectory of the spinal screw 130. Images from the imaging system 250 may be processed at the computing device 210 to determine the 3D position and orientation of the surgical device 100 relative to the spine 10 at that approximate position and orientation, prior to bone alteration. Assuming that the patient is still, as expected during such surgery, and using appropriate imaging modality so as not to have to move the patient (e.g., C-arm), images of the spine 10 and of the surgical device 100 may be obtained, and correlated to tracking data from the computing device 210 at the instant of the imaging. This may be achieved by appropriate synchronization techniques (e.g., using internal clock or time stamps). This allows the CAS system 200 to locate the surgical device 100 and the spine 10 in the same coordinate system (a.k.a., referential system, frame of reference, etc), for subsequently tracking the surgical device 110 relative to the spine 10, in position and orientation, with the movements of the surgical device 110 being tracked by the sensor 220. The above may require some additional steps by the computing device 210, some of which may include obtaining or generating 3D models of the spine 10 using for example a bone atlas, or preoperative models of the spine 10 specific to the patient, merging existing models of the spine to the images, etc. In some embodiments, the images from the imaging system 250 may be processed at the computing device 210 to determine the anticipated 3D position and orientation of the spinal screw 130 relative to the spine 10, using geometrical relations described above. The 3D position and orientation of the surgical device 100 may thus be determined based on the known configuration of the surgical device 100 (e.g., the length of the attachment member 110, the position of the trackable member 120 on the attachment member 110 if present, and/or the configuration of the trackable member 120, the coupling configuration between the attachment member 110 and the screw 130, etc.), whereby it is possible to determine the position and trajectory of the screw 130. This may be done during the placement of the screw 130 into a vertebra. Consequently, data from the optical sensor(s) 220 may be processed by the computing device 210 to obtain position information of the attachment member 110, for example via the trackable member 120. Based on the position information of the trackable member 120 and the 3D position of the surgical device 100 as determined from the images, the 3D position of the surgical device 100 relative to the spine 10 may be tracked by the CAS system 200 throughout surgery. Assuming that the patient does not move, the position of the surgical device 100 relative to the spine 10 may be determined at the CAS system 200 based on the data from the optical sensor(s) 220. The surgical device 100 may then be used to implant the spinal screw 130 into a vertebra 14 of the spine 10. This arrangement may cause the surgery to be less invasive, notably because an operator does not need to physically see the trajectory of the screw 130, relying instead on the combination of imaging and tracking. For this purpose, the surgical device 100 may be coated with radiopaque material to have a high contrast definition when imaged by the imaging system 250.

The CAS system 200 may thus determine the position and orientation of the surgical device 100 relative to the spine 10 as the spinal screw 130 is implanted in a vertebra 12. As another possibility, once the spinal screw 130 is inserted in a pedicle 14 of a vertebra 12 with the surgical device 100, the position and orientation of the surgical device 100 relative to the spine 10 may be determined using the geometrical relations described above.

The 3D position and orientation of the surgical device 100 relative to the spine 10 may be registered (e.g., stored at the computing device 210) in order to create a position and orientation reference of the surgical device 100. The registration of the 3D position and orientation may occur prior to or after implantation of the spinal screw 130 in a vertebra 12 of the spine 10. The registered 3D position and orientation of the surgical device 100, and/or the spinal screw 130, may provide a position and orientation reference used during subsequent steps of the surgery. For example, the screw 130 may be a first inserted screw for the surgery and using the position and orientation reference of the screw 130, the position and orientation of subsequent implants (e.g., screws, other devices, etc.) may be determined and displayed on the display device 230.

The CAS system 200 may be configured to generate a 3D coordinate system X-Y-Z relative to the spine 10. Data from the optical sensor(s) 220 may be processed by the computing device 210 to obtain the position and orientation information of the surgical device 100, for example via the trackable member 120. Based on the 3D position and orientation of the surgical device 100 relative to the spine 10 as determined from the images of the imaging system 250, a 3D coordinate system X-Y-Z relative to the spine 10 may be generated at the computing device 210.

The CAS system 200 may be configured to track the spine 10 once the spinal screw 130 is implanted in a vertebra 12 of the spine 10. Accordingly, the CAS system 200 may be configured to track the spine 10 once the surgical device 100 is coupled to the spine 10 via the spinal screw 130. The CAS system 200 may be configured to identify and/or track the position and orientation of the spine 10 based on the position and orientation reference of the surgical device 100 (or spinal screw 130) for example via the position information of the trackable member 120 and. In some embodiments, the position and orientation of the spine 10 may be identified and tracked by the CAS system 200 in the 3D coordinate system X-Y-Z. More specifically, data from the optical sensor(s) 220 may be processed by the computing device 210 to identify the position and orientation of the surgical device 100 and hence the spine 10 in the 3D coordinate system X-Y-Z. This may provide the surgeon with an accurate representation of the position and orientation of the spine 10 during the surgery.

The CAS system 200 may be configured to identify and/or track one or more surgical tools and/or implants. The surgical tool(s) and/or implant(s) may be identified and/or track based on the position and orientation reference of the surgical device 100 (or spinal screw 130). For example, the surgical tool(s) and/or implant(s) may be identified and tracked by the CAS system 200 in the 3D coordinate system X-Y-Z. More specifically, data from the optical sensor(s) 220 may be processed by the computing device 210 to identify a surgical tool (or an implant) and the 3D position and orientation of the surgical tool (or the implant) in the 3D coordinate system X-Y-Z may be determined. The position and orientation of the surgical tool (or the implant) relative to the images of the spine 10 may be displayed on the display device 230. This may provide the surgeon with an accurate representation of the position and orientation of the surgical tool (or the implant) relative to the spine 10.

In some embodiments, the surgical device 100 may be moved along the vertebrae as multiple surgical screws are implanted, while performing the identification and/or tracking described herein. The attachment member 110 may be configured to decouple from an implanted surgical screw in order to be used for implanting another surgical screw. Accordingly, multiple surgical screws may be implanted in multiple pedicles of the vertebrae with the surgical device 100. The surgical device 100 may have a release mechanism adapted to cause the attachment member 110 to decouple for an implanted surgical screw. The surgical device 100 may be slid off of the screw 130, for example. The surgical device 100 may then be used to implant another surgical screw. The surgical device 100 may be used with one or more implants used for interconnecting one or more vertebrae, for example, such as one or more of the implants described in U.S. Pat. No. 7,107,091, the contents of which are hereby incorporated by reference. The imaging of the patient's spine may be updated each time a new surgical screw is implanted, may occur continuously during the surgery, or may be updated at any regular interval or irregularly. Based on the updated imaging, the CAS system 200 may be able to update the 3D position and orientation of the surgical device 100 relative to the spine 10 and continue the identification and/or tracking described herein. Imaging may not need to be updated when multiple spinal screws are implanted with the surgical device 100, for example, when the patient does not move.

In some embodiments, the CAS system 200 may be configured to create an anatomical model with either pre-operative images and/or with intra-operative images of the patient, which is displayed on the display device 230 during the surgery. The anatomical model may be used in place or in conjunction with the images from the imaging system 250 to determine the position and orientation reference. The anatomical model of the spine 10, the intra-operative images of the spine 10, the position and orientation of the surgical device 100 and/or the position and orientation of the surgical tool(s) and/or implant(s) may be displayed on the display device 230 during the surgery.

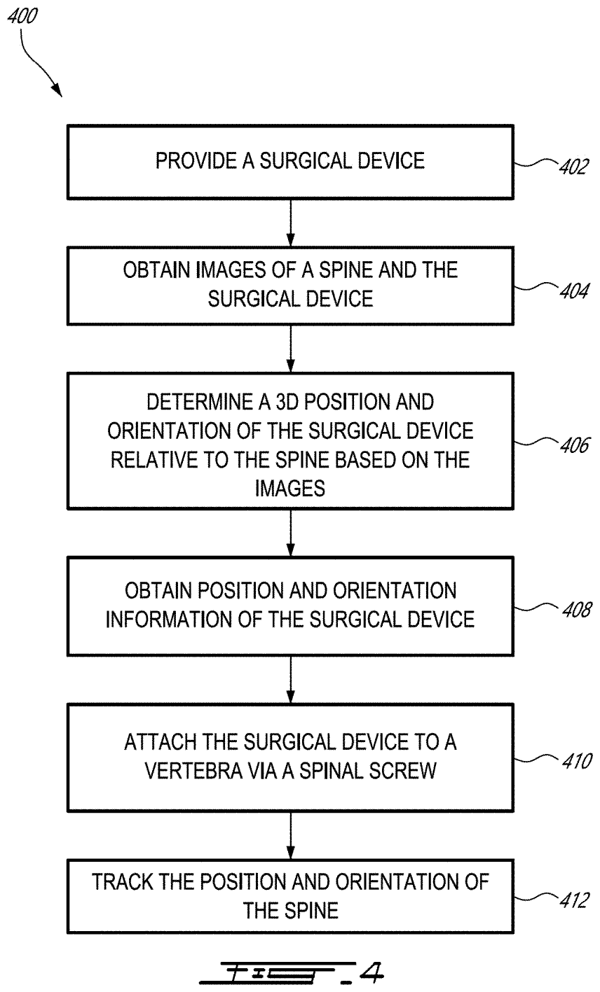

With additional reference to FIG. 3, there is shown a flow diagram illustrating an example of a computer-assisted surgical process 300 performed with the surgical device 100 and the CAS system 200. At step 302, a surgeon makes an initial incision for spinal surgery on a patient. This initial incision may be a minimally invasive incision. At step 304, the surgeon estimates a position and/or orientation of the pedicle 14 of a given vertebra 12 of the spine 10 of the patient using the surgical device 100, and uses a tool, just as the surgical device 100, in an approximate desired position and trajectory of a spinal screw. It may be possible to have a robotic arm, such as robotic arm 240, hold the surgical device 100 in place in the desired position and orientation. At step 306, images of the patient are obtained at the CAS system 200. The images of the patient may be obtained with the image system 250. The obtained images may include X-ray images obtained with a C-arm. In some embodiments, the registration of the 3D position and orientation of the surgical device 100 relative to spine 10 and/or any planning (e.g., an anatomical model generated with pre-operative images) may occur at step 306. The registration may be automatic and entails a combination of the instant images and tracking output from the CAS system 200, to locate the spine 10 and the surgical device 100 in a 3D common coordinate system, as explained above.

In an embodiment, the automatic registration includes using the anatomical model generated with pre-operative images and/or modelling techniques, such as a 3D model of the spine 10, and registering the 3D model of the spine 10 with the images from the image system 250. For example, U.S. Pat. No. 9,826,919, incorporated herein by reference, describes a method and system for generating a display of a tracked object relative to a vertebra, and includes the combination of radiographic images with models. As another possibility, the automatic registration includes a Digitally Rendered Radiographs (DRR) technique, by which a 3D pre-operative model is matched to the 2D images from the image system 250. As part of the image processing performed by the registration, the geometry of the surgical device 100, or like pointer tool, may be taken into consideration. The geometry of the surgical device 100 or like pointer tool may be known pre-operatively, and the geometry of the device 100 is additional data that may be used in the sizing and scaling computations. Other steps may be required, though optionally, such as the registration of prominent features of vertebrae, such as the spinous process, by the operator or robotic arm 420, to contribute to or confirm the registration of the spine 10 in the referential system. Consequently, the registration may not be fully automatic, as some verification steps or additional data gathering steps may be required. Upon completion, the registration provides the known position and orientation of the spine 10 in the virtual referential system tracked by the CAS system 200, such that subsequent tracking of devices by the CAS system 200 is relative to the spine 10.

Once the 3D position and orientation of the surgical device 100 relative to spine 10 is registered, the position and orientation of surgical device 100 may be tracked by the CAS system 200 with additional use of the optical sensor(s) 220, the tracking being for instance continuous and in real-time. The position and orientation of surgical device 100, or any other instrument may thus be tracked during movement of the surgical device 100 using the tracking of the trackable member 120 and the geometrical relation between the trackable member 120, if present, the surgical device 100 and spinal screw 130. At step 308, the surgical device 100 is used to insert into the patient the spinal screw 130. This may involve the tracking of a drilling tool 308A or any other tool to make a hole at a desired trajectory in the vertebra 12. This may entail that the patient has not moved from registration to positioning of the screw 130. For instance, at step 308, the surgical device 100 may be navigated by controlling the robotic arm 240 to move the position and orientation of the surgical device 100 or drilling tool 308A into a position for inserting the spinal screw 130. This may occur in collaborative mode as well, with a user manipulating the surgical device 100 and spinal screw 130, with navigation data provided via the GUI 230, for example. The robotic arm 240 may then lock the surgical device 100 in a desired trajectory for the spinal screw 130. At step 312, the spinal screw 130 is inserted. For example, after the drilling tool 308A is navigated into the desired position and orientation as per a pre-operative plan or based on operator decisions, a hole for the spinal screw 130 may be drilled and tapped in a vertebra 12, and in particular a pedicle 14, per the pre-operative plan. The spinal screw 130 may then be implanted in the hole. At step 312, one or more dilators 310A are placed over the surgical device 100. The dilator 310A may be a tube, such as with a tapered end, that may be used to push or pull soft tissue away from the hole in the vertebra. The dilator 310A may be slid onto a drill bit, drill pin of the drilling tool 308A as a possibility. The surgical device 100 may be used to drill and/or implant the spinal screw 130 into the vertebra 12. This may occur with the dilator 310A in place. Once the surgical device 100 is attached to the vertebra 12 via the spinal screw 130, the position and orientation of the spine 10 may be tracked by the CAS system 200, with reference to the surgical device 100 remaining connected to the vertebra 12. The position and orientation of the spine 10 may be tracked using the tracking of the trackable member 120 and the geometrical relation between the trackable member 120, the surgical device 100 and spinal screw 130, or directly by tracking the surgical device 100 if tracking modality permits. Similarly, once the surgical device 100 is attached to the vertebra 12 via the spinal screw 130, the position and orientation of one or more surgical tools and/or implants may be tracked by the CAS system 200. For example, additional spinal screws 130 are added to other vertebrae 14, along some of the actions taken in steps 302-312 described above, but with or without imaging as per step 304, as the tracking of the surgical device 100 anchored to a vertebra 14 may provide the tracking accuracy for the subsequent alterations steps to be performed. The steps of the process 300 may vary depending on practical implementations, as the order of the steps may vary and/or some steps may be omitted and/or combined. For example, the images of patent at step 306 may occur at one or more different steps of the process 300. By way of another example, the other of step 302 and 304 may be reversed. Other modifications are possible. Hence, in a variant, the surgical device 100 as connected to a vertebra 14 via a spinal screw 130 may serve as tracking reference for the tracking of other tools (e.g., the drilling tool 308A) performing alterations on other vertebrae 14.

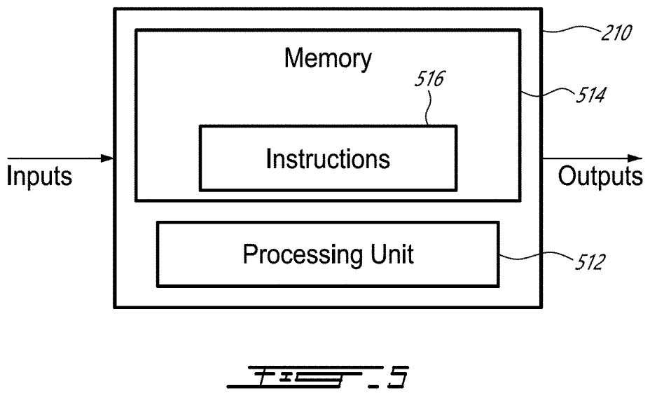

With reference to FIG. 4, there is shown a flowchart illustrating an example method 400 for a computer-assisted surgical process. The method 400 may be at least in part implemented by the computing device 210 associated with the CAS system 200. It should be appreciated that aspects of the process 300 and the method 400 may be combined, as one or more the steps of the method 400 may occurring during one or more steps of the process 300.

Step 402 of the method 400 includes obtaining a surgical device 100 including an attachment member 110 adapted for coupling to a spinal screw 130. The attachment member 110 may have a trackable member 120 coupled to the attachment member 110, or may be trackable without a trackable member 120. The surgical device 100 may configured as described elsewhere in this document. Other tools may be obtained such as a registration pointer-like tool or drilling tool having a configuration similar to that of the surgical device 100. For example, such tool may have an elongated shape with a central axis that emulates the surgical device 100 with the screw 130. The tool may be the surgical device 100 without screw 130.

Step 404 of the method 400 includes obtaining, at a CAS system 200, images of the spine 10 and the surgical device 100 or like tool. The images may be obtained from the imaging system 250. The images of the spine 10 may be X-ray images providing both a lateral and posterior or anterior perspective of the spine 10, such as those provided by a C-arm. In the image, the spine 10 is spatially correlated to the surgical device 100 or like tool. In a variant, the surgical device 100 or like tool is positioned and oriented at an estimated drilling trajectory within a given vertebra.

Step 406 of the method 400 includes determining, at the CAS system 200, a 3D position and orientation of the surgical device 100 or like tool relative to the spine 10 from the images of the spine 10 and the surgical device 100, in a referential system (e.g., a X,Y,Z coordinate system). This may include a determination of the 3D position and orientation of the attachment member 110, the trackable member 120, and/or the spinal screw 130 relative to the spine 10. The 3D position and orientation of the surgical device 100 may be used to provide a position and orientation reference of the surgical service 100, i.e., to set the position and orientation of a trackable tool relative to the spine 10 in the referential system. The 3D position and orientation of the attachment member 110, the trackable member 120 (if present), and/or the spinal screw 130 may be used to provide a position and orientation reference. From that point on, real-time tracking of any tool, including the surgical device 100, may be performed, for instance by the CAS system 200.

Step 408 of the method 400 includes obtaining, at the CAS system 200, position and orientation information of the surgical device 100, as the surgical device 100 moves relative to the spine 10, or of other surgical devices such as a drill. Stated differently, devices such as the surgical device 100 may be moved relative to the spine 10, and the position and orientation of the tool may be output relative to the spine 10. Obtaining the position and orientation information of the surgical device 100 may include obtaining position information of the trackable member 120. The position and orientation information of the surgical device 100 may be determined from the obtaining position information of the trackable member 120. The position information may be provided by an optical system including the one or more optical sensors 220 or may be determined at the CAS system 200 based on data obtained by one or more optical sensors 220. In some embodiments, the method 400 includes tracking the position and orientation of the surgical device 100 based on the position and orientation information of the surgical device 100 and the 3D position and orientation of the surgical device 100 as determined per step 406. The position and orientation of the surgical device 100 may be tracked using the tracking of the trackable member 120—or the tracking of the attachment member 110 directly—and the geometrical relation between the trackable member 120 if present, the surgical device 100 and spinal screw 130. The tracking may be continuous, or may be in continuous periods.

Step 410 of the method 400 includes attaching the surgical device 100 to a vertebra 12 of a spine 10 via the spinal screw 130 implanted in the vertebra 12. In some embodiments, the surgical device 100 is attached to the spinal screw 130 after the spinal screw 130 is implanted in the vertebra 12. In some embodiments, the spinal screw 130 is implanted in the vertebra 12 with the surgical device 100 having the spinal screw 130 coupled thereto. In an embodiment, step 410 includes tracking tool tapping a hole in the vertebra 12 using trajectory angles obtained by the tracking of step 408, prior to securing the surgical device 100 to the vertebra 14 via the spinal screw 130. Step 408 may occur continuously during step 410, with step 410 being guided by the data provided in step 408. A drilling tool 308A (FIG. 3) may be used and tracked for drilling the vertebra on the desired trajectory. The robotic arm 240 may be controlled to preserve a desired trajectory. The trajectory may be as planned, or as decided by an operator (e.g., surgeon) based on the navigation output of step 408. Once the hole is drilled, a dilator (e.g., 310A, FIG. 3) may space surrounding soft tissue away from the hole, for the spinal screw 130 to then be screwed in via the surgical device 100. The surgical device 100 may then remain anchored during surgery to define a trackable reference of the spine 14.

Step 412 of the method includes tracking, at the CAS system 200, the spine 10 based on the position and orientation information of the surgical device 100 (e.g., position information of trackable member 120) and the 3D position and orientation of the surgical device 100. The optical sensor(s) 220 (or the optical system) may be used to sense the position and orientation of the surgical device 100 and the spine 10 may be tracked based on this information of the surgical device 100. The position and orientation of the spine 10 may be tracked using the tracking of the trackable member 120 and the geometrical relation between the trackable member 120, the surgical device 100 and spinal screw 130, and the known position and orientation of the spinal screw 130 implanted in the spine 10.

In some embodiments, the method 400 includes tracking, at the CAS system 200, one or more surgical tools or implants relative to the spine 10 based on the 3D position and orientation of the surgical device 100 (e.g., the position and orientation reference) and the position and orientation information of the surgical device 100 (e.g., the position information of trackable member 120). The optical sensor(s) 220 (or optical system) may be used to sense the surgical tool(s) or implant(s) and the position of the surgical tool(s) or implant(s) relative to the spine 10 may accordingly be determined. For example, additional spinal screws 130 are added to other vertebrae 14, but with or without imaging as per step 404, as the tracking of the surgical device 100 anchored to a vertebra 14 may provide the tracking accuracy for the subsequent alterations steps to be performed. The surgical device 100 as connected to a vertebra 14 via a spinal screw 130 may serve as tracking reference for the tracking of other tools (e.g., the drilling tool 308A) performing alterations on other vertebrae 14. The robotic arm 240 may assist in holding the surgical device 100 during such other alterations. In an embodiment, the tracking steps of 408 and 412 are performed by the continuous operation of the sensor(s) 220.

In an embodiment, the devices and methods described herein may render the spinal surgery less invasive, as the use of the spinal screw(s) 130 as an attachment for a trackable device (e.g., the surgical device 100 via its attachment member 110, with or without the trackable member 120) may limit the incision to the vertebra (with dilators optionally present to assist). Moreover, because of the accuracy of the surgical device 100 remaining on the spinal screw 130, smaller incisions may be made at other vertebra(e) 14 for alterations and installation of other spinal screws 130. The surgical device 100, or other tool, with or without the trackable member 120, becomes a trackable reference.

The method 400 may further comprise generating a 3D coordinate system X-Y-Z relative to the spine 10 in a manner as described elsewhere in this document. Accordingly, the tracking of the spine 10 and/or of the surgical tool(s) or implant(s) may occur in the 3D coordinate system X-Y-Z. The tracking information may be output for display on the display device 230. For example, the position and orientation of the spine 10 and/or the position and orientation of the surgical tool(s) or implant(s) relative to the spine 10 may be displayed. The steps of the method 400 may vary depending on practical implementations, as the order of the steps may vary and/or some steps may be omitted and/or combined.

It should be appreciated that by performing the surgery with the surgical device 100 and/or the CAS system 200 that the invasiveness of the surgery may be reduced or minimized as additional surgical openings for a reference and/or tracking device may be omitted.

While the embodiments and examples described above relate to use of the surgical device 100 and the CAS system 200 in a spinal surgery, the device 100, the CAS system 200, the process 300 and the method 400 may be adapted for any other suitable surgery where a screw is inserted into a bone and tracking of a bone, surgical tools and/or implants are desired.

With reference to FIG. 5, at least in part, the process 300 and/or the method 400 may be implemented by a computing device 210, comprising a processing unit 512 and a memory 514 which has stored therein computer-executable instructions 516. The processing unit 512 may comprise any suitable devices configured to implement at least in part the process 300 or the method 400 such that instructions 516, when executed by the computing device 210 and/or other programmable apparatus, may cause the functions/acts/steps performed as part of the process 300 and/or the method 400 as described herein to be executed. The processing unit 512 may comprise, for example, any type of general-purpose microprocessor or microcontroller, a digital signal processing (DSP) processor, a central processing unit (CPU), a graphical processing unit (GPU), an integrated circuit, a field programmable gate array (FPGA), a reconfigurable processor, other suitably programmed or programmable logic circuits, or any combination thereof.

The memory 514 may comprise any suitable known or other machine-readable storage medium. The memory 514 may comprise non-transitory computer readable storage medium, for example, but not limited to, an electronic, magnetic, optical, electromagnetic, infrared, or semiconductor system, apparatus, or device, or any suitable combination of the foregoing. The memory 514 may include a suitable combination of any type of computer memory that is located either internally or externally to device, for example random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like. Memory 514 may comprise any storage means (e.g., devices) suitable for retrievably storing machine-readable instructions 516 executable by processing unit 512.

The methods and systems described herein may be implemented in a high level procedural or object oriented programming or scripting language, or a combination thereof, to communicate with or assist in the operation of a computer system, for example the computing device 210. Alternatively, the methods and systems described herein may be implemented in assembly or machine language. The language may be a compiled or interpreted language. Program code for implementing the methods and systems may be stored on a storage media or a device, for example a ROM, a magnetic disk, an optical disc, a flash drive, or any other suitable storage media or device. The program code may be readable by a general or special-purpose programmable computer for configuring and operating the computer when the storage media or device is read by the computer to perform the procedures described herein. Embodiments of the methods and systems may also be considered to be implemented by way of a non-transitory computer-readable storage medium having a computer program stored thereon. The computer program may comprise computer-readable instructions which cause a computer, or in some embodiments the processing unit 512 of the computing device 210, to operate in a specific and predefined manner to perform the functions described herein.

Computer-executable instructions may be in many forms, including program modules, executed by one or more computers or other devices. Generally, program modules include routines, programs, objects, components, data structures, etc., that perform particular tasks or implement particular abstract data types. Typically the functionality of the program modules may be combined or distributed as desired in various embodiments.

### Examples

The following examples can each stand on their own, or can be combined in different permutations, combinations, with one or more of other examples.

Example 1 is a method for spine tracking in computer-assisted surgery, the method comprising: obtaining, at a computer-assisted surgical system, at least one image of at least part of the spine and at least one surgical device; determining, at the computer-assisted surgical system, a three-dimensional position and orientation of the at least one surgical device relative to the spine from the at least one image to create a referential system; tracking, at the computer-assisted surgical system, the at least one surgical device altering a first vertebra of the spine for attachment of a spinal screw to the first vertebra, in the referential system; and tracking, at the computer-assisted surgical system, the spine in the referential system with a trackable reference attached to the spinal screw of the first vertebra.

In Example 2, the subject matter of Example 1 includes, wherein tracking the spine in the referential system includes tracking the at least one surgical device altering at least a second vertebra of the spine.

In Example 3, the subject matter of Example 2 includes, wherein tracking the at least one surgical device altering at least the second vertebra of the spine is performed without additional obtaining at least one image.

In Example 4, the subject matter of Examples 1 to 3 includes, wherein tracking the spine in the referential system includes tracking the trackable reference being a surgical device used to screw the spinal screw in the first vertebra.

In Example 5, the subject matter of Examples 1 to 4, including controlling a robotic arm to hold the trackable reference fixed.

In Example 6, the subject matter of Examples 1 to 5 includes, wherein obtaining at least one image includes obtaining at least one image with a C-arm.

In Example 7, the subject matter of Examples 1 to 6 includes, wherein obtaining at least one image includes generating a model of the spine using the at least one image.

In Example 8, the subject matter of Example 7 includes, wherein generating the model includes using an existing bone model with the at least one image.

In Example 9, the subject matter of Examples 1 to 8 includes, wherein tracking the at least one surgical device includes outputting a GUI display of the at least one surgical device relative to the spine.

Example 10 is a system for spine tracking in computer-assisted surgery, the system comprising: a processing unit; and a non-transitory computer-readable memory having stored thereon program instructions executable by the processing unit for: obtaining at least one image of at least part of the spine and at least one surgical device; automatically registering a three-dimensional position and orientation of the at least one surgical device relative to the spine from the at least one image to create a referential system; tracking the at least one surgical device altering a first vertebra of the spine for attachment of a spinal screw to the first vertebra, in the referential system; and tracking the spine in the referential system with a trackable reference attached to the spinal screw of the first vertebra.

In Example 11, the subject matter of Example 10 includes, wherein tracking the spine in the referential system includes tracking the at least one surgical device altering at least a second vertebra of the spine.

In Example 12, the subject matter of Example 11 includes, wherein tracking the at least one surgical device altering at least the second vertebra of the spine is performed without additional obtaining at least one image.

In Example 13, the subject matter of Examples 10 to 12 includes, wherein tracking the spine in the referential system includes tracking the trackable reference being a surgical device used to screw the spinal screw in the first vertebra.

In Example 14, the subject matter of Examples 10 to 13, including controlling a robotic arm to hold the trackable reference fixed.

In Example 15, the subject matter of Examples 10 to 14 includes, wherein obtaining at least one image includes obtaining at least one image with a C-arm.

In Example 16, the subject matter of Examples 10 to 15 includes, wherein obtaining at least one image includes generating a model of the spine using the at least one image.

In Example 17, the subject matter of Example 16 includes, wherein generating the model includes using an existing bone model with the at least one image.

In Example 18, the subject matter of Examples 10 to 17 includes, wherein tracking the at least one surgical device includes outputting a GUI display of the at least one surgical device relative to the spine.

In Example 19, the subject matter of Examples 10-18, including the at least one surgical device.

In Example 20, the subject matter of Example 19 includes, wherein the at least one surgical device includes a drilling tool.

In Example 21, the subject matter of Examples 19 to 20 includes, wherein the at least one surgical device includes a surgical device having an attachment tool for connection to the spinal screw.

In Example 22, the subject matter of Example 21 includes, wherein the attachment tool includes a rotor in a hollow tube for rotatably receiving a connector on the spinal screw.

In Example 23, the subject matter of Examples 10-22, further including at least one sensor device for tracking the at least one surgical device.

In Example 24, the subject matter of Example 23, further including at least one trackable member secured to the at least one surgical device and trackable by the at least one sensor device.

In Example 25, the subject matter of Examples 10-24, further including at least one imaging system for obtaining the image.

In Example 26, the subject matter of Example 14, further including the robotic arm.

Example 27 is an assembly for spine tracking in computer-assisted surgery, the assembly comprising: a spinal screw having a connector; a surgical device including an attachment member for coupling to the spinal screw, and a trackable member coupled to the attachment member, the trackable member including at least one detectable element for being tracked in three-dimensional space by a computer-assisted surgical system, thereby allowing tracking position and orientation of a spine by the computer-assisted surgical system when the attachment member is coupled to the spinal screw implanted in a vertebra of the spine.

In Example 28, the subject matter of Example 27 includes, wherein the connector has a pair of elongated tabs.

In Example 29, the subject matter of Examples 27 and 28 includes, wherein the attachment member includes a tube for housing the pair of elongated tabs.

In Example 30, the subject matter of Example 29 includes, wherein the attachment member includes a rotor within the tube.

In Example 31, the subject matter of Example 30 includes, wherein the rotor has flats for coupling engagement with the elongated tabs.

In Example 32, the subject matter of Examples 30 and 31 including a handle for rotating the rotor.

The above description is meant to be exemplary only, and one skilled in the art will recognize that changes may be made to the embodiments described without departing from the scope of the invention disclosed. Still other modifications which fall within the scope of the present invention will be apparent to those skilled in the art, in light of a review of this disclosure.

Various aspects of the methods, systems and devices described herein may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments. Although particular embodiments have been shown and described, it will be obvious to those skilled in the art that changes and modifications may be made without departing from this invention in its broader aspects. The scope of the following claims should not be limited by the embodiments set forth in the examples, but should be given the broadest reasonable interpretation consistent with the description as a whole.

What is claimed is:

1. A system for spine tracking in computer-assisted surgery, the system comprising:
a processing unit; and
a non-transitory computer-readable memory having stored thereon program instructions executable by the processing unit for:
obtaining at least one image of at least part of the spine and at least one surgical device;
automatically registering a three-dimensional position and orientation of the at least one surgical device relative to the spine from the at least one image to create a referential system;
tracking the at least one surgical device altering a first vertebra of the spine for attachment of a spinal screw to the first vertebra, in the referential system; and
tracking the spine in the referential system with a trackable reference attached to the spinal screw of the first vertebra.

2. The system according to claim 1, wherein tracking the spine in the referential system includes tracking the at least one surgical device altering at least a second vertebra of the spine.

3. The system according to claim 2, wherein tracking the at least one surgical device altering at least the second vertebra of the spine is performed without additional obtaining at least one image.

4. The system according to claim 1, wherein tracking the spine in the referential system includes tracking the trackable reference being a surgical device used to screw the spinal screw in the first vertebra.

5. The system according to claim 1, including controlling a robotic arm to hold the trackable reference fixed.

6. The system according to claim 1, wherein obtaining at least one image includes obtaining at least one image with a C-arm.

7. The system according to claim 1, wherein obtaining at least one image includes generating a model of the spine using the at least one image.

8. The system according to claim 7, wherein generating the model includes using an existing bone model with the at least one image.

9. The system according to claim 1, wherein tracking the at least one surgical device includes outputting a GUI display of the at least one surgical device relative to the spine.

10. The system according to claim 1, including the at least one surgical device.

11. The system according to claim 10, wherein the at least one surgical device includes a drilling tool.

12. The system according to claim 1, wherein the at least one surgical device includes a surgical device having an attachment tool for connection to the spinal screw.

13. The system according to claim 12, wherein the attachment tool includes a rotor in a hollow tube for rotatably receiving a connector on the spinal screw.

14. The system according to claim 1, further including at least one sensor device for tracking the at least one surgical device.

15. The system according to claim 14, further including at least one trackable member secured to the at least one surgical device and trackable by the at least one sensor device.

16. The system according to claim 1, further including at least one imaging system for obtaining the image.

17. The system according to claim 5, further including the robotic arm.

18. An assembly for spine tracking in computer-assisted surgery, the assembly comprising:
a spinal screw having a connector,
a surgical device including an attachment member for coupling to the spinal screw, and
a trackable member coupled to the attachment member, the trackable member including at least one detectable element for being tracked in three-dimensional space by a computer-assisted surgical system, thereby allowing tracking position and orientation of a spine by the computer-assisted surgical system when the attachment member is coupled to the spinal screw implanted in a vertebra of the spine.
